(19) United States
(12) Patent Application Publication
(10) Pub. No.: US 2021/0322727 A1
(43) Pub. Date: Oct. 21, 2021

(54) CATHETER WITH ELECTRICALLY-ACTUATED ARTICULATION

(71) Applicant: Enlight Medical Technologies (Shanghai) Co., Ltd., Shanghai (CN)

(72) Inventor: Jie Yu Rao, Plano, TX (US)

(73) Assignee: Enlight Medical Technologies (Shanghai) Co., Ltd., Shanghai (CN)

(21) Appl. No.: 17/233,822

(22) Filed: Apr. 19, 2021

Related U.S. Application Data
(60) Provisional application No. 63/012,764, filed on Apr. 20, 2020.

(51) Int. Cl. A61M 25/01

(52) U.S. Cl. CPC A61M 25/0158; A61M 2205/0266; A61M 25/0138; A61M 25/0147

(57) ABSTRACT

The present teachings provide an articulation catheter configured to be activated by electric energy. Specifically, one aspect of the present teachings provide a catheter body with a central axial lumen for delivering a medical device, and a plurality of peripheral lumens for articulation wires. The articulating wires join an energy source that controls the activation. The articulation wires are made of a conductive portion and an articulating portion made of shape memory material. The conductive portion of the articulating wire transfers electric energy to the articulating portion of the articulating wire. The articulating portion of the articulating wire transitions into its pre-programmed shape upon resistance heating caused by the electric energy received.

### CROSS REFERENCE TO RELATED APPLICATIONS

This application claims priority to and the benefit of U.S. Provisional Patent Application Ser. No. 63/012,764, entitled “CATHETER WITH ELECTRICALLY-ACTUATED ARTICULATION,” filed on Apr. 20, 2020, the entirety of which is incorporated herein by reference.

### FIELD

The present teachings relate to an articulation catheter which is configured to be activated by electric energy.

### BACKGROUND

The ability to remotely manipulate instruments and tools is of wide interest. Such technique is desirable in situations where the targeted site is difficult or unsafe for direct access. In percutaneous medical procedures, the catheter movement is typically controlled by a physician from outside of the body. Such movement includes extending the catheter forward and retracting it backward.

Steerable catheters are also well known for both diagnostic and therapeutic applications. These special designed catheters allow the physician to further control the movement of the distal tip of the catheter, for example, bending and/or twisting in a predefined direction. Many attempts have been made to design catheters with improved steerability. For example, U.S. Pat. No. 3,557,780 to Sato; U.S. Pat. No. 5,271,381 to Ailinger et al.; U.S. Pat. No. 5,922,146 to Alotta et al.; and U.S. Pat. No. 6,270,453 to Sakai, all describe endoscopic instruments with one or more flexible portions that could be bent by actuation of a single set of wires. These wires are actuated from the proximal control mechanism, specifically by rotating pinions and/or a pulley mechanism (Sato), manipulating knobs (Ailinger et al.), and/or a steerable arm (Alotta et al.). U.S. Pat. No. 5,922,147 to Boury et al. further discloses a steerable catheter having four wires that run within the catheter wall. Each wire terminates at a different part of the catheter. The proximal end of the wires extend loosely from the catheter so that the physician may pull them. The physician is able to shape and thereby steer the catheter by selectively placing the wires under tension.

Although each of the catheter designs described above achieve the goal of remotely steering the distal catheter tip from outside of the patient body, the motion range for the distal tip of these catheter, however, are generally limited. In one part, this is because that typically only one single cable set are employed in connecting links or segments of the steerable elements. Since the distal links or segments bend together as a unit or units, as a result, independent movement at each link or segment is not possible. In addition, steerably catheter with each wire must be separately pulled to achieve the motion, for example as described in Boury, could be laborious to use. All knob and pulley mechanisms would require a significant amount of training for a physician to be proficient in maneuvering such device.

In addition, limitations in the current design include limited range of motion which makes it difficult to negotiate acute angles (e.g., in the rectosigmoid colon), creating patient discomfort and increasing the risk of trauma to surrounding tissues.

Consequently, there is a need for a device with enhanced remote maneuverability. It is highly desirable for such device to be able to navigate complex anatomy, efficiently and advance and deploy surgical and diagnostic instruments with precision, reduce trauma to surrounding tissues, and minimize patient discomfort. It is also highly desirable for such device to provide an intuitive and facile user interface and therefore reduce any possibility physician error.

### SUMMARY

One aspect of the present teachings provides an articulation catheter system comprising of a catheter body, at least one articulating wire, and an energy source. The catheter body has a proximal end, a distal end, an elongated axial central lumen. The elongated axial central lumen extends from the proximal end to the distal end. The at least one articulating wire extends parallel to the elongated axial central lumen of the catheter body. The at least one articulating wire has a conductive portion and an articulating portion. A proximal end of the articulating wire connects to the energy source. The conductive portion of the articulating wire conducts electric energy from the energy source to the articulating portion of the articulating wire. The articulating portion of the articulating wire changes into a pre-programmed shape upon resistance heating when activated by the energy.

Another aspect of the present teachings provides is that the catheter body of the articulating catheter system further comprises of an articulating portion and a non-articulating portion. In one embodiment of the present teaching, the articulating portion of the catheter body is position at a distal portion of the catheter body. Another aspect of the present teachings provides that the portion of the articulating catheter body further comprises of a plurality of segment linked together by articulating wire.

Another aspect of the present teachings provides that the catheter body of the articulating catheter system further comprises of multiple articulating portions. And the multiple articulating portions are separated by a non-articulating portion in between adjacent articulating portions along the catheter body.

Another aspect of the present teachings provides that the catheter body of the articulating catheter system of further comprises at least a pair of peripheral lumens extending parallel to the longitudinal axial central lumen. The at least one articulating wire of the articulating catheter system is configured to extending inside the first of the at least one peripheral lumens distally, and extending inside a second of the at least one peripheral lumens proximally after making a U-turn at the distal end of the catheter body.

Another aspect of the present teachings provides that the catheter body of the articulating catheter system further comprises 2-4 articulating wires configured to deflect different portions of the catheter body.

Another aspect of the present teachings provides that the at least one articulating wire of the articulating catheter system is configured to be bonded to the catheter body. In one exemplary embodiment the at least one articulating wire of the articulating catheter system is configured to be bonded to an external luminal surface of the catheter body. In another embodiment, the at least one articulating wire of the articulating catheter system is configured to be bonded to the catheter body through wire extrusion.

Another aspect of the present teachings provides that the articulating portion of the articulating wire is made of shape memory wire. In another embodiment, the articulating portion of the articulating wire is made of shape memory polymer film.

Another aspect of the present teachings provides that the energy source of the articulating catheter system further includes a control which regulates the current applying to the articulating wire. In one embodiment, the articulating wire of the articulating catheter system is configured to deflect greater at a higher current.

### BRIEF DESCRIPTION OF THE DRAWINGS

FIG. 1 is a perspective view of an exemplary catheter system in accordance with one embodiment of the present teachings.

FIG. 2A-2E are perspective views of distal end view of the catheter system in accordance with various embodiments of the present teachings

FIG. 3A-3B are perspective views of the distal end configuration of the catheter body in accordance with various embodiments of the present teachings.

FIG. 4 is a perspective view of an exemplary embodiment of the articulating portion of the catheter system in accordance with one embodiment of the present teachings.

FIG. 5 is a perspective view of an exemplary articulating catheter body with multiple articulating portions in accordance with one embodiment of the present teachings.

FIG. 6 is a perspective view of an exemplary catheter system with the articulating portion activated in accordance with one embodiment of the present teachings.

### DETAILED DESCRIPTION

Certain specific details are set forth in the following descriptions and figures to provide an understanding of various embodiments of the present teachings. Those of ordinary skill in the relevant art would understand that they can practice other embodiments of the present teachings without one or more of the details described herein. Thus, it is not the intention of the applicant(s) to restrict or in any way limit the scope of the appended claims to such details. While various processes are described with reference to steps and sequences in the following disclosure, the steps, and sequences of steps should not be taken as required to practice all embodiments of the present teachings.

As used herein, the term “lumen” means a canal, a duct, or a generally tubular space or cavity in the body of a subject, including veins, arteries, blood vessels, capillaries, intestines, and the like. The term “lumen” can also refer to a tubular space in a catheter, a sheath, a hollow needle, a tube, or the like.

As used herein, the term “proximal” shall mean close to the operator (less into the body) and “distal” shall mean away from the operator (further into the body). In positioning a medical device inside a patient, “distal” refers to the direction relatively away from a catheter insertion location and “proximal” refers to the direction relatively close to the insertion location.

As used herein, the terms “radially outward” and “radially away” means any direction which is not parallel with the central axis. For example, considering a cylinder, a radial outward member could be a piece of wire or a loop of wire that is attached or otherwise operatively coupled to the cylinder that is oriented at an angle greater than 0° relative to the central longitudinal axis of the cylinder.

As used herein, the term “wire” can be a strand, a cord, a fiber, a yarn, a filament, a cable, a thread, or the like, and these terms may be used interchangeably.

As used herein, the term “sheath” may also be described as a “catheter” and, thus, these terms can be used interchangeably.

Unless otherwise specified, all numbers expressing quantities, measurements, and other properties or parameters used in the specification and claims are to be understood as being modified in all instances by the term “about.” Accordingly, unless otherwise indicated, it should be understood that the numerical parameters set forth in the following specifications and appended claims are approximations. At the very least and not as an attempt to limit the application of the doctrine of equivalents to the scope of the claims, numerical parameters should be read in light of the number of reported significant digits and the application of ordinary rounding techniques.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third, etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence.

The term “and/or” where used herein is to be taken as specific disclosure of each of the two specified features or components with or without the other. For example “A and/or B” is to be taken as specific disclosure of each of (i) A, (ii) B, and (iii) A and B, just as if each is set out individually herein.

The term “diameter” where used herein to describe a non-circular geometry is to be taken as the diameter of a hypothetical circle approximating the geometry being described. For example, when describing a cross-section, such as the cross-section of a component, the term “diameter” shall be taken to represent the diameter of a hypothetical circle with the same cross-sectional area as the cross-section of the component being described.

The terms “major axis” and “minor axis” of a component where used herein are the length and diameter, respectively, of the smallest volume hypothetical cylinder which can completely surround the component.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination. For example, it will be appreciated that all features set out in any of the claims (whether independent or dependent) can be combined in any given way.

The present teachings relate to an articulation catheter shaft design with the ability to be activated remotely. According to one embodiment, such catheter shaft having a single lumen with a plurality of articulation points. According to one embodiment of the present teaching, the disclosed catheter configuration offers excellent distal flexibility, an outstanding proximal pushability and torquability, superior external lubricity, and superior internal surface lubricity.

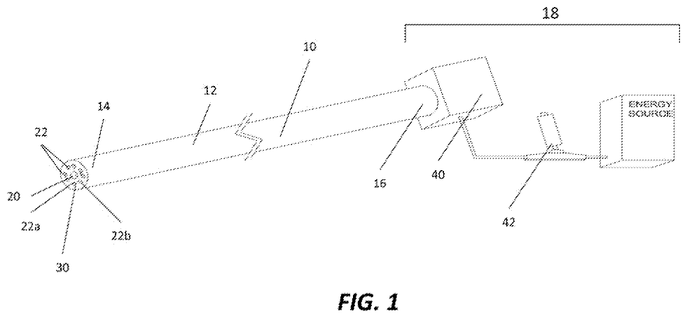

FIG. 1 illustrates one embodiment of an articulation catheter (10). As illustrated, the catheter (10) has a longitudinal catheter body (12), a central axial lumen (20) extending longitudinally throughout the catheter body (12), and a plurality of peripheral lumens (22) also extending longitudinally throughout the catheter body (12). The proximal end (16) of the articulating catheter (10) connects to a control system (18), which, for example, includes a liquid management system, as well as energy source that initiates the articulation of the catheter. According to one embodiment of the present teaching, as shown in FIG. 1, the plurality of the peripheral lumens (22) extends parallel to the central axial lumen (20), and are positioned radially away from the central axial lumen (20). An articulating wire (30) extends distally inside a first peripheral lumen (22B) from the proximal end toward the distal end. When reaching outside of the distal end of the peripheral lumen (22B), the articulating wire (30) makes a U-turn, and further extends proximally through a second peripheral lumen (22C) adjacent to the first peripheral lumen (22B) from its distal end to its proximal end. Both ends of the articulating wire (30) extends proximally outside of the peripheral lumen (22) and are connected to an energy source. The proximal end (16) of the catheter body (12) connects to a sealing block for liquid management, and the proximal end of the articulating wires (30) extending through the sealing block and connects the energy source.

According to one embodiment of the present teachings, the catheter body (12) has a uniform diameter throughout its entire length. In one embodiment, the catheter body (12) has a diameter of 0.040-0.400 inches and an overall length of 5-110 inches. In one embodiment, the non-articulating portion of the catheter body could be 5-100 inches long and the articulating portion of the catheter body would 0.5-10 inches long. In another embodiment, the diameter of the catheter body (12) varies along its longitudinal length. In one exemplary embodiment, the diameter of the catheter body (12) gradually reduces from its proximal end (16) toward its distal end (14). In another exemplary embodiment, a proximal portion of the catheter body (12) has a first larger diameter, a distal portion of the catheter body (12) has a second smaller diameter, and the diameter of the intermediate portion in between the proximal and distal portions of the catheter body (12) which gradually reduces from the first diameter to the second diameter. One skilled in the art should recognize that the catheter body (12) could have more diameter profile across multiple portions of its entire length, thus exemplary embodiment shown and described above should not be viewed as limiting to the scope of the present teaching.

According to one embodiment of the present teaching, the catheter body (12) could have a unity construction such as manufactured via multiple lumen extrusion technique. In an alternative embodiment, the catheter body (12) could have multiple-step construction. For example, the central axial lumen (20) of the catheter body (12) could be made of one tubing, and the multiple peripheral lumens made of separate tubing; all tubings are then composited together through known techniques in the industry. According to another embodiment, the catheter body (12) could further include other layers, such as mesh layer made of metal wires to improve kink resistance, and/or additional surface layer in order to improve surface smoothness for better vascular insertion. One skilled in the art should understand exemplary embodiment described herein should not be viewed as limiting to the scope of the present teaching.

According to one embodiment, the catheter body (12) could be made from a flexible polymer such as Nylon, PEBAX, Polyurethane, silicone, or other material known in the field. According to one embodiment of the present teachings, a radioopaque marker is used to make the catheter body (12) visible using radiographic imaging equipment such as X-ray, magnetic resonance, ultrasound or other imaging techniques. Markers may be applied to any part of the catheter body (12) by sewing, adhering, swaging, and etc. The radioopaque markers may be formed of tantalum, tungsten, platinum, irridium, gold, alloys of these materials, or other materials that are known to those skilled in the art. In an alternative embodiment, the catheter body (12) could be made of materials blended with radiopaque compound, such as Barium Sulfate, Bismuth, Tungsten, so that the entire length of the catheter body (12) could be visible through imaging equipment.

According to one embodiment of the present teachings, only the distal portion of the catheter could articulate once activated. In an alternative embodiment, any portion of the catheter can articulate upon activation. According to one embodiment of the present teachings, the articulating portion of the catheter is made of the same material as the rest portion of the catheter. In an alternative embodiment, the articulating portion of the catheter is made of a different material from the rest portion of the catheter. Yet, in another embodiment of the present teaching, the material used for the articulating portion of the catheter is configured to stretch and compression easily in order to accommodate the shape change of the portion of the catheter.

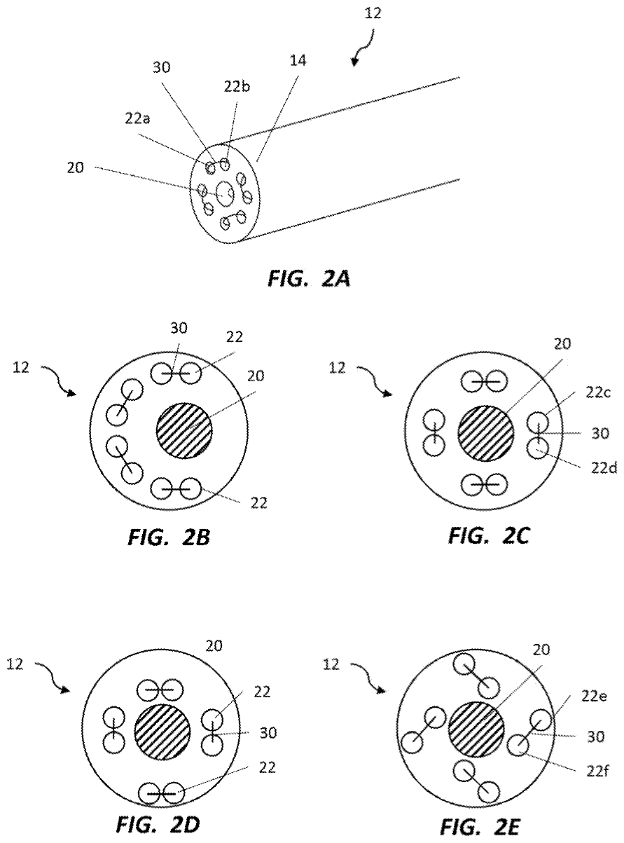

Now referring back to FIG. 1, according to one embodiment, the central axial lumen (20) of the catheter body (12) is configured to be used as a conduit for delivering medical implants such as stents, valve replacements, or implants for areas other than the heart and circulatory system. The central axial lumen (20) of the catheter body could also be used as conduit for surgical instruments percutaneous access to a treatment location such as a RF electrode, or a guide wire. According to one embodiment, the central axial lumen (20) is positioned at the radial center of the elongated catheter body (12), such as shown in FIG. 2A. In an alternative embodiment, the central axial lumen (20) could be positioned at a position off radial center of the elongated catheter body (12) (such as shown in the cross section view of FIG. 2B). According to one embodiment of the present teaching, the central axial lumen (20) is 0.010-0.380 inches diameter. And 5-95% of the diameter of the overall diameter of the catheter body.

The peripheral lumens (22) are positioned evenly around the central axial lumen (20) as shown in FIG. 2A. One articulating wire (3) extending distally and then proximally, after making a U turn outside of the distal end (14) of the catheter body, through a pair of the peripheral lumens (22a, 22b). According to one embodiment of the present teaching, a pair of peripheral lumens (22a, 22b) are configured to be closely adjacent to each other so that one articulating wire (30) extends distally inside a first one (22c), makes a U-turn, and then extends proximally inside the adjacent second one (22d). And each pair of peripheral lumens are positioned relatively further away from the other pairs, such as shown in FIG. 2C. For illustration purpose, four pairs of the peripheral lumens (22) are shown in figures throughout present disclosure. According to one embodiment of the present teachings, there are 2-6 pairs of peripheral lumens (22) positioned around the central axial lumen (20). According to one embodiment, all peripheral lumens (22) are of equal radial distance from the central axial lumen (20) as shown in FIG. 2A. According to an alternative embodiment, at least one peripheral lumen (22d) is positioned at a different radial distance from the central axial lumen (20) comparing to other peripheral lumens (22) as shown in FIG. 2D. According to one embodiment, all peripheral lumens (22) are of equal distance from each other as shown in FIG. 2A. In one embodiment, the two peripheral lumens (22) with the same articulating wire (30) are of the same radial distance from the central axial lumen (20) as shown in FIGS. 2A and 2C. In another embodiment, the two peripheral lumens (22e, 22f) with the same articulating wire (30) are of the different radial distance from the central axial lumen (20) as shown in FIG. 2E.

In one embodiment, the diameter of the peripheral lumen is 0.04-0.4000 inches. According to another embodiment, the diameter of the peripheral lumen is 1-100% of the central axial lumen (20). In another embodiment, the diameter of the all peripheral lumens (22) are the same. In another embodiment, at least one peripheral lumen has a different diameter than the rest of the peripheral lumens (22). According to an alternative embodiment, the central axial lumen (20) is sized with a larger diameter than all the peripheral lumens (22). In another embodiment, the central axial lumen (20) is sized with the same diameter as at least one peripheral lumen.

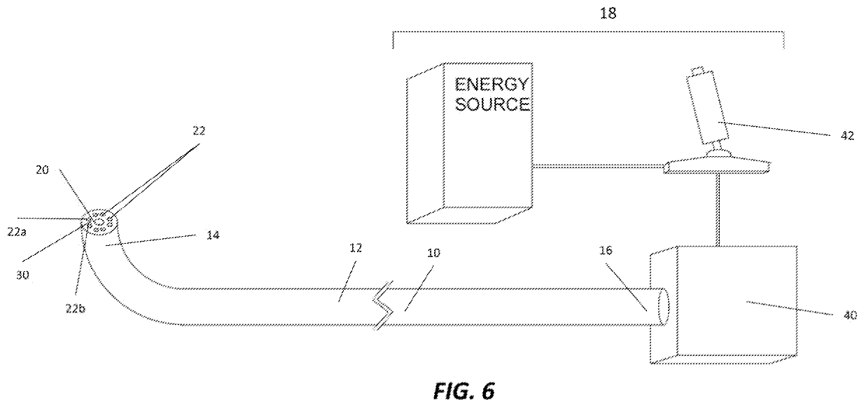

Now referring back to FIG. 1, according to one embodiment of the present teaching, the articulating wire (30) is configured to extend along the longitudinal lumens of a pair of peripheral lumens (22a, 22b) as shown in FIG. 1. Both ends of the articulating wire (30) extend outside of the proximal end of the catheter body (12), through the liquid sealing block and connects to an energy source. According to one embodiment, the wire has a conductive portion and an articulating portion. The conductive portion of the wire is configured to transmit energy to the articulating portion of the wire. Once activated by energy, the articulating portion of the wire is activated with resistance heating, and changes its profile into pre-programmed shape. According to one embodiment of the present teachings, the articulating portion of the wire is positioned at the distal portion (14) of the catheter body (12), as such as exemplary embodiment shown in FIG. 6. Once activate, the shape change of the articulating wire leads to the profile change of the distal portion of the catheter. According to an alternative embodiment, the articulating portion of the wire could be positioned at any portion of the catheter body (12).

According to one embodiment of the present teaching, the articulating portion of the wire (30) could be made of shape memory which would change its shape once heated to a pre-defined temperature. For example, the articulating portion of the wire (30) could be made of copper-aluminum-nickel and nickel-titanium (NiTi). Specifically, the articulating portion of the wire (30) could be made of nitinol material. According to one embodiment, the articulating portion of the wire (30) is pre-shaped at a pre-defined temperature through means known by those skilled in the art. Thus, when the energy transmits through the articulating portion, the wire is heated to a pre-defined temperature, and then automatically transitions into its pre-defined shape, which transforms the catheter body (12) into a controlled bend/or deflection. In another embodiment, the conductive portion of the wire (30) could be made of any conductive metal or alloy. In an alternative embodiment, the conductive portion of the wire (30) could be other made of other type of conductive elements, such as a conductive thin film. According to one embodiment of the present teachings, the articulating wire (30) could have an overall diameter of 0.002-0.100 inches. In an alternative exemplary embodiment, the conductive thin film could have a thickness of 1-1000 microns.

Now referring back to FIG. 1, the articulating wire (30) extends through the pair of the peripheral lumens (22), with both ends of the articulating wire (30) connecting to an energy source. In one embodiment, the articulating wire (30) is anchored at multiple axial locations along the longitudinal peripheral lumens (22). Such intermittent anchoring configuration allows the catheter body (12) to stretch and/or compress when deflects upon articulation. For example, the articulating wire (30) would be attached to 2-8 different locations inside each of the peripheral lumen (22) between the distal and the proximal ends (14, 16) of the catheter body (12). One skilled in the art should understand that what has been described is merely an exemplary embodiment where the articulating wire (30) could be anchored to more or less than 4 locations along the peripheral lumen.

In an alternative embodiment, the articulating wire could be built into the catheter body via many known manufacturing method, such as wire extrusion method. In such way, the articulating wire is fed into the extruder that makes the catheter body. In another embodiment, the articulating wire could be positioned outside of the elongated catheter body. According to one embodiment of the present teaching, the articulating element and/or wire could be bond along the external longitudinal surface of the catheter bond via thermal, chemical or mechanical means. Articulating catheter built with such methods, wire extrusion, or bonded to the external longitudinal surface of the catheter body, would not have peripheral lumens as described herein.

Now referring back to FIG. 1, the proximal end (16) of the catheter body (12) terminates at a liquid sealing block (40). According to one embodiment, the liquid sealing block (40) could be a circuit board, or a solid polymer block that provides for a hermetic seal for all open lumens at the proximal portion of the catheter (10). Continue referring to FIG. 1, all proximal ends of the articulating wires (30) connect to a control (42), which controls electrical current and/or voltage from energy source. When energy is applied, the conductive portion of the articulating wire transfer the current to the articulating portion of the wire. Due to resistance heating, the articulating portion of the wire is then heat up, and triggers a pre-programmed shape change. As a result, the articulating portion of the catheter body (12) responds to shape change of the articulating wire, and deflects accordingly. In one embodiment, the control (42) regulates the current and/or voltage that applies to the articulating wire (30). In one embodiment, the more voltage and/or current applies to the articulating wire (30), the more deflection it results.

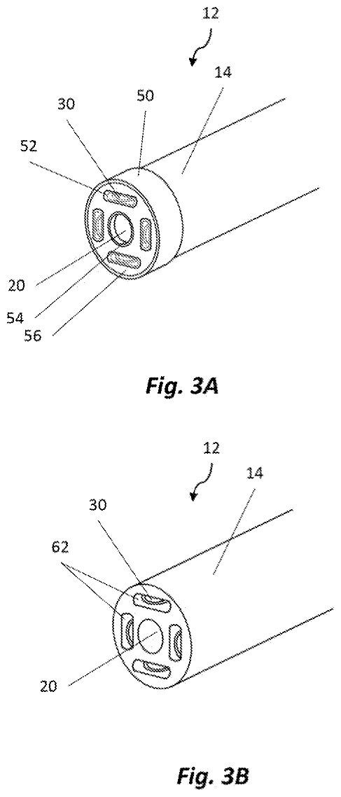

According to one embodiment of the present teachings, in order to prevent the articulating wire (30) from protruding outside of the distal end (14) of the catheter body (12), such as the exemplary embodiment shown in FIG. 2A, special feature could be constructed at the distal end (14) of the catheter body (12). FIG. 3A shows a distal cap (50) fixed to the distal end (14) of the catheter body (12). As shown in FIG. 3A, a plurality of slots (52) on the distal cap (50) is configured to be positioned over each pair of peripheral lumen with the same articulating wire (30). The distal cap (50) further includes a center opening (54) to be positioned over the distal end (14) of the central axial lumen (20) of the catheter body (12). With the distal cap (50), a portion of the articulating wire (30), specially, the portion forming the U-turn that is outside of the distal end (14) of the catheter body (12) is recess proximally from the distal end (56) of the distal cap (50). According to one embodiment of the present teachings, the distal cap (50) could be made of polymer materials such as Nylon, PEEK, Polycarbonate, or ABS, or metals such as stainless steel, Titanium, Nitinol, or Elgiloy. In another embodiment, the distal cap (50) could be bonded to the distal end (14) of the catheter body (12) through known technique in the field, such as thermal, chemical or mechanical bonding.

FIG. 3B shows another design where a portion of the articulating wire, specially, the portion of the articulating wire (30) forming the U-turn, is recess proximally from the distal end (14) of the catheter body (12). Specifically, recess slots (62) is constructed at the distal end (14) of the catheter body (12) among the pair of the peripheral lumens (22) with the same articulating wires (30). One skilled in the art should know that other designs could be incorporated to the distal end (14) of the catheter body (12) in order to achieve a relative flat/smooth distal end in order to prevent the distal end of the catheter body from damaging surrounding tissue. Thus, exemplary embodiments illustrated and disclosed herein should not be viewed at limiting.

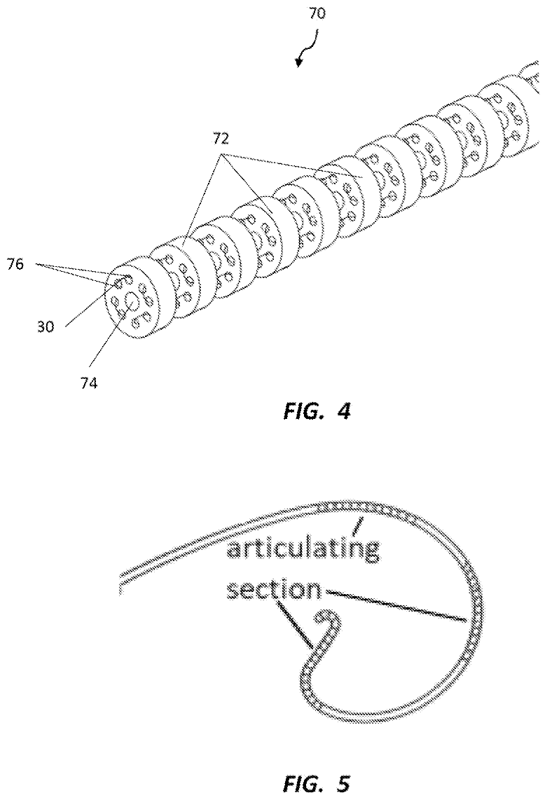

FIG. 4 illustrates another embodiment of the articulating portion (70) of the catheter body (12) according to one embodiment of the present teachings. According to one embodiment, the articulating portion (70) is made of a plurality of segments (72). Similar to the exemplary embodiment described above in reference to FIG. 1, each segment (72) of the articulating portion (70) has a central axial opening (74) and multiple pairs of peripheral openings (76). As the plurality of segments (72) align together forming an articulating portion (70) of the catheter body (12), the central axial openings (74) of the each segments (72) aligned together forming a central axial lumen (20), and the pairs of peripheral openings (76) aligned together forming pairs of peripheral lumens (22). According to one embodiment of the presenting teaching, similar to what has been described in accordance with FIG. 1, one articulating wire (30) extends through the pair of peripheral lumens (22) with both ends of the wire extending outside of the proximal end (16) of the catheter body. Additionally, all segments (72) are anchored to the articulating wire (30). The application of an electrical voltage or current to the articulating wire (30) causes corresponding relative movements of the segmented articulating portion (70) of the catheter body (12). The segmented articulating portion (70) of the catheter body (12) provides an advantage of enhanced flexibility to accommodate catheter deflection during articulating. One skilled in the art should understand that although not specifically defined, each segment is configured to maintain a spaced apart relationship relative to the other segments (72) in order to allow easy deflection.

According to one embodiment of the present teachings, the catheter body (12) could be made entirely of segments (72). According to another embodiment, only a distal portion of the catheter body (12) is made of a plurality of segments (72), as shown in FIG. 4. In another embodiment, the catheter body (12) could have segmented section (70) at any location of the along the longitudinal catheter body (12). In one embodiment, only one segmented articulating portion (70) is incorporated into a catheter body (12). In another embodiment, more than one segmented articulating portion (70) could be incorporated into one catheter body (12) according to the specific application of the catheter system, such as shown in FIG. 5. According to one embedment, the segments (72) that make up the catheter body (12) have uniform size. In an alternative embodiment, the segments (72) vary in size, i.e. diameter or length according to the specific application of the catheter system.

Now referring to FIG. 6, where the distal portion of the catheter body (12) deflects when the articulating wire (30) is activated by energy source, according to one embodiment of the present teachings. As shown in the figure, as the articulating wire (30) is energized and heated by Joule resistance heating, it transitions into its pre-defined shape. For example, as shown in FIG. 5, the distal portion of the catheter body (12) deflects. According to one embodiment, the angle of deflection to the longitudinal axis of the catheter body could be 0-225 degrees. The deflection tip length could be 0.50-10.0 inches long.

According to one embodiment, the articulating mechanism of the present teachings includes having multiple articulating wires (30) that extend along the entire longitudinal length of the catheter body (12) as shown in FIG. 1. According to another embodiment, the articulating mechanism of the present teachings could also including having articulating wire extend to certain location along the longitudinal catheter body where articulating movement is desired, for example, around the middle portion of the catheter body. In one embodiment, the catheter design could have a first articulating wire extending throughout the entire length of the catheter body, while a second articulating wire extending only to a location proximal for the distal end of the catheter body. And the two articulating wires are programmed to deflect toward two different or even opposite direction, so as to accommodate the tortious path within human vasculature. In another embodiment, more than one articulating wire could be used, each terminating at a different axial location along the catheter body.

The present invention discloses an articulation catheter with its articulating mechanism activated by means of electric energy. Since, such articulating mechanism can be initiated by a simpler control such as a foot pedal, it would give a clinician greater freedom in other task, such as implant delivery/deployment and etc. However, one skilled in the art should understand that the presently disclosed energy-deflecting mechanism may be combined with mechanical articulating mechanism in order to satisfy the needs of a variety of surgical procedures.

The foregoing description and accompanying drawings set forth a number of examples of representative embodiments at the present time. Various modifications and alternative designs will become apparent to those skilled in the art in light of the foregoing teachings without departing from the spirit hereof, or exceeding the scope hereof, which is indicated by the following claims rather than by the foregoing description. All changes and variations that fall within the meaning and range of equivalency of the claims are to be embraced within their scope. Additionally, the present teachings are capable of other embodiments or of being practiced or carried out in various other ways. Also, it is to be understood that the phraseology and terminology employed herein is for the purpose of description and should not be regarded as limiting.

Unless otherwise defined, all technical and scientific terms used herein have the same meaning as commonly understood by one of ordinary skill in the art to which this present teachings belong. Methods and materials similar or equivalent to those described herein can be used in the practice or testing of the present teachings. In case of conflict, the patent specification, including definitions, will control. In addition, the materials, methods, and examples are illustrative only and not intended to be limiting.

We claim:

1. An articulation catheter system comprising:
a catheter body having a proximal end, a distal end, an elongated axial central lumen extending from the proximal end to the distal end;
at least one pair of peripheral lumens extending parallel to the elongated axial central lumen, wherein the at least one pair of peripheral lumens are positioned radially away from the elongated axial central lumen;
at least one articulating wire extending inside a first peripheral lumen from a proximal end toward a distal end, making a U-turn, then extending inside a second peripheral lumen from a distal end toward a proximal end, with both ends of the at least one articulating wire extending outside of the proximal ends of the at least one pair of peripheral lumens; wherein the at least one articulating wire has a conductive portion and an articulating portion;
an energy source connecting to the proximal ends of the articulating wire,
wherein the conductive portion of the articulating wire conducts electric energy from the energy source to the articulating portion of the articulating wire, and wherein the articulating portion of the articulating wire is configured to change into a pre-programmed shape upon resistance heating when activated by the energy.

2. The articulating catheter system of claim 1, wherein the catheter body further comprises an articulating portion and a non-articulating portion, and wherein the articulating portion of the catheter body is configured to deflect according to the shape change of the articulating wire when activated by the energy.

3. The articulating catheter system of claim 2, wherein the articulating portion of the catheter body is positioned at a distal portion of the catheter body.

4. The articulating catheter system of claim 2, wherein the articulating portion of the catheter body further comprises a plurality of segments linked together by the articulating wire, wherein each segment has a central axial opening aligning with the elongated axial central lumen of the non-articulating portion of the catheter body, and multiple pairs of peripheral openings aligning with the peripheral lumens of the non-articulating portion of the catheter body.

5. The articulating catheter system of claim 1, wherein the catheter body further comprises multiple articulating portions separated by a non-articulating portion in between two articulating portions.

6. The articulating catheter system of claim 1, wherein the catheter body further comprises multiple pairs of peripheral lumens extending parallel to the longitudinal axial central lumen.

7. The articulation catheter system of claim 1 further comprising multiple articulating wires extending through the multiple pairs of peripheral lumens, wherein each articulating wire is configured to deflect different portions of the catheter body.

8. The articulating catheter system of claim 1, wherein the at least one articulating wire of the articulating catheter system is configured to bond along the peripheral lumen of the catheter body.

9. The articulating catheter system of claim 1, wherein the energy source of the articulating catheter system further includes a control which regulates the current applying to the articulating wire.

10. The articulating catheter system of claim 1, wherein the articulating wire of the articulating catheter system is configured to deflect to a greater amount at a higher current.

11. The articulating catheter system of claim 1, wherein the catheter body further comprises a distal cap fixed to the distal end of the catheter body, wherein the distal cap has at least one slot configured to be positioned over the at least one pair of peripheral lumen with the same articulating wire.

12. The articulating catheter system of claim 1, wherein the catheter body further comprises a distal recess, where a portion of the U-turn articulating wire positioned inside the distal recess.

13. An articulation catheter system comprising:
a catheter body having a proximal end, a distal end, an elongated axial central lumen extending from the proximal end to the distal end;
at least one articulating wire bonds along the external longitudinal surface of the catheter body, wherein the at least one articulating wire extends from the proximal end toward the distal end of the catheter body, making a U-turn, then extending from the distal end toward the proximal end of the catheter body, with both ends of the at least one articulating wire extending the proximal ends of the catheter body; wherein the at least one articulating wire has a conductive portion and an articulating portion;
an energy source connecting to the proximal ends of the articulating wire,
wherein the conductive portion of the articulating wire conducts electric energy from the energy source to the articulating portion of the articulating wire, and wherein the articulating portion of the articulating wire is configured to change into a pre-programmed shape upon resistance heating when activated by the energy.

14. The articulating catheter system of claim 14, wherein the catheter body further comprises an articulating portion and a non-articulating portion, and the articulating portion of the catheter body is configured to deflect according to the shape change of the articulating wire when activated by the energy.

15. The articulation catheter system of claim 14 further comprising multiple articulating wires bonding along an external longitudinal surface of the catheter body, wherein each articulating wire is configured to deflect different portions of the catheter body.
